(19) United States
(12) Patent Application Publication
(10) Pub. No.: US 2025/0098940 A1
(43) Pub. Date: Mar. 27, 2025

(54) ROTARY ARTICULATING CHOLANGIOSCOPE WITH STABILIZING OVERTUBE

(71) Applicant: GYRUS ACMI, INC. D/B/A OLYMPUS SURGICAL TECHNOLOGIES AMERICA, Westborough, MA (US)

(72) Inventors: Anthony R. Pirozzi, Raleigh, NC (US); Richard K. Lessig, Macungie, PA (US)

(21) Appl. No.: 18/729,044

(22) PCT Filed: Jan. 17, 2023
(86) PCT No.: PCT/US2023/060763

§ 371 Date: Jul. 15, 2024

Related U.S. Application Data
(60) Provisional application No. 63/267,038, filed on Jan. 21, 2022.
(60) Provisional application No. 63/358,757, filed on Jul. 6, 2022.

(51) Int. Cl. A61B 1/00; A61B 1/273

(52) U.S. Cl. CPC A61B 1/00135; A61B 1/00087; A61B 1/00147; A61B 1/273

(57) ABSTRACT

An endoscopy system can include or use an endoscopic overtube, sized and shaped for insertion into a patient esophagus towards a patient duodenum. The overtube can include and access lumen providing an inner diameter to receive and pass a cholangioscope therethrough. The distal portion of the overtube can include or use a side port sized and shaped to permit lateral exit of a distal portion of the cholangioscope. Also, the overtube can include a ramp arranged to re-direct the distal portion of the cholangioscope from a longitudinal direction in the access lumen toward the lateral exit of the side port.

### CLAIM OF PRIORITY

This application claims priority to U.S. Provisional Application Ser. No. 63/267,038, filed on Jan. 21, 2022, and U.S. Provisional Application Ser. No. 63/358,757, filed on Jul. 6, 2022, which are each incorporated by reference herein in their entirety, and the benefit of priority of each is claimed herein.

### TECHNICAL FIELD

This document describes generally, among other things, an endoscope for cholangioscopy or other procedures, and more particularly to systems and methods for a cholangioscope with a rotatable articulation mechanism and an overtube for guiding the cholangioscope towards a patient duodenum.

### BACKGROUND

Endoscopes can be used in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices or biological matter collection devices for sampling or treating an anatomical region, and providing suction passageways for collecting fluids (e.g., saline or other preparations or bodily fluids), among other procedures. Examples of such anatomical region can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

In certain endoscopy, the distal portion of the endoscope can be configured for supporting and orienting another therapeutic device. In some systems, two endoscopes can be used. A first, parent endoscope can be used for guiding a second, child endoscope inserted therein and directing the second endoscope, such as using an elevator located at the distal end of the first endoscope. Such systems can be helpful in guiding an endoscope to anatomic locations that are relatively distant from an insertion location.

Peroral cholangioscopy is an endoscopy technique that permits direct endoscopic visualization, diagnosis, and treatment of various disorders of patient biliary and pancreatic ductal system using miniature endoscopes and catheters inserted through the accessory port of a duodenoscope. Peroral cholangioscopy can be performed by using a dedicated cholangioscope that is advanced through the accessory channel of a duodenoscope, as used in Endoscopic Retrograde Cholangio-Pancreatography (ERCP) procedures. ERCP is a technique that combines the use of endoscopy and fluoroscopy to diagnose and treat certain problems of the biliary or pancreatic ductal systems, including the liver, gallbladder, bile ducts, pancreas, or pancreatic duct. In ERCP, a cholangioscope (here acting as an auxiliary scope, or a “daughter” scope) can be attached to and advanced through a working channel of a duodenoscope (here acting as a primary scope, or a “parent” scope). In ERCP, biliary cannulation can be achieved directly with the tip of the cholangioscope alone or passed over a guidewire such as to promote ease and control of certain procedures. A tissue retrieval device can be inserted through a channel of the cholangioscope such as to address biological matter (e.g., gallstones, bill duct stones, cancerous tissue) or to manage a stricture or blockage in bile duct.

### SUMMARY

This document describes generally, among other things, an endoscope for endoscopic procedures (which can refer generally to include endoscopic, arthroscopic, laparoscopic or similar minimally-invasive procedures), which may include a rotary mechanism providing rotary-assisted articulation to move a distal tip of a cholangioscope and an overtube for guiding the cholangioscope into anatomic locations within the body. The endoscope can include or use a handheld portion and an insertion portion for partial insertion into an orifice of or incision in a patient such as for assessing, diagnosing, or treating a target within the patient.

The present inventors have recognized challenges with conventional medical devices, and in particular endoscopes and duodenoscopes. Such challenges can include, among other things, 1) the difficulty in navigating endoscopes to difficult to reach anatomic locations, 2) the increased time and associated cost of navigating an endoscope to an incorrect location, and 3) the possibility of potential tissue effects of impacting sensitive tissue with an endoscope. Such problems can be particularly present in duodenoscopy procedures in which an auxiliary scope (also referred to as daughter scope, or cholangioscope) can be attached and advanced through the working channel of a “main scope” (also referred to as a mother scope or duodenoscope). The present document describes an endoscopic technique that can enable access of a cholangioscope within a patient, such as using only one scope and obviating a need for another parent scope to be used with the cholangioscope for certain procedures. For example, a single scope may be inserted through a patient's mouth, down the esophagus, through the stomach, into the duodenum, through the ampulla of vater, and into the bile ducts or the pancreatic ducts. Devices and methods described herein can help obviate a need for using a duodenoscope for navigation of, e.g., upper gastrointestinal anatomy to the ampulla of vater and then advancing a separate cholangioscope through the working channel of the duodenoscope to cannulate patient anatomy.

For example, the proximal portion of the scope can include an external handle for a physician or other user to hold and support the proximal portion during insertion of the distal portion into the patient. The external handle may include electronic or other controls for operating the cholangioscope, such as can include an articulation mechanism and a rotary mechanism.

The present document describes a cholangioscope that can include a camera, a light source, a port for irrigation or guidewire passage, one or more accessory ports for insertion of accessory devices such as diagnostic or therapeutic devices, or a combination thereof. The diagnostic or therapeutic devices that may be inserted into the accessory port may comprise, for example, an ablation catheter, a Through the Scope (TTS) dilation balloon, a TTS stent, a TTS cytology brush, a biopsy forceps, a lithotripsy probe/filter, or a combination thereof. The endoscope may be used with electronics for providing signal processing, such as can include algorithmic techniques, artificial intelligence (AI) techniques, and narrow-band imaging (NBI) and other imaging modes for contrast and providing more information in the imaging. Endoscopes described herein can be interchangeably referred to as a cholangioscope, in reference to other cholangioscopy techniques, but can also refer generally to a single endoscope for accessing anatomy inferior to a pylorus of a stomach of a patient without the need to be guided by a parent endoscope. As such, cholangioscopes described herein need not be limited by other associations with cholangioscope devices configured for use with a duodenoscope.

In an example of the present approach, the rotary mechanism can be coupled to the handle portion of the cholangioscope. A separate articulation mechanism can be operatable via handle portion such as to enable planar articulation of a distal tip of the cholangioscope. The rotary mechanism can enable the physician to rotate the plane of articulation of the distal tip of the cholangioscope, such as by up to 360 degrees (e.g., 180 degrees clockwise and 180 degrees counterclockwise) relative to its neutral orientation. The combination of independent rotary and articulation controls can help allow for precise direction of the distal tip of the cholangioscope such as to help better navigate the anatomical structures of the patient anatomy below the pylorus of the stomach.

The cholangioscope can be passed through an overtube. The overtube can be semi-rigid, such as providing rigidity sufficient to help manipulate the shape of the patient anatomical route. For example, the overtube can be placed along the patient anatomical route to help straighten one or more bends included in a natural disposition of the patient anatomy. For example, the rigidity can help provide a splinting of the pylorus such as to help pull the anatomy into a straighter conformation. The overtube can also have flexibility sufficient to pass through the pylorus and to direct the cholangioscope into a patient duodenum.

The overtube can include an aperture at a side of a distal end of the overtube. The aperture can guide the cholangioscope passing through the overtube such as to turn into the biliary anatomy passively, thereby helping to obviate the need for the physician to utilize the articulation mechanism and rotary mechanism to make the turn. The passive turn can help reduce mechanical stress and wear on the cholangioscope.

In an example, the overtube can include or use comprise an overtube stabilizer. The overtube stabilizer can include a stabilizing balloon located in an area toward the distal tip of the overtube.

Techniques described herein can help eliminate the difficulty of the physician otherwise having to use two separate scopes (e.g., the duodenoscope and cholangioscope). For example, cholangioscopes described herein can be operated by one physician and can provide precision of movement of the distal portion of the cholangioscope with the addition of the rotary mechanism to allow 360-degree rotation of the single plane movement provided by the articulation mechanism. The stabilization balloon can help reduce inadvertent movement of various devices during an endoscopic procedure.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a schematic diagram of endoscopy system including imaging and control system, a cholangioscope, and an endoscopic overtube.

FIG. 1B depicts a step within a sequence of events to illustrate the rotation action of the cholangioscope.

FIG. 1C depicts a step within a sequence of events to illustrate the rotation action of the cholangioscope.

FIG. 1D depicts a step within a sequence of events to illustrate the rotation action of the cholangioscope.

FIG. 1E depicts an example of a handle portion including a knob for controlling the rotating portion.

FIG. 2A depicts a portion of an example of a cholangioscope.

FIG. 2B illustrates an example of an electrosurgical system in use by a physician.

FIG. 3A depicts a distal portion of an example of an endoscopic overtube positioned in a patient duodenum.

FIG. 3B depicts a magnified view of a distal portion of an example of an endoscopic overtube 110 positioned in a patient duodenum.

FIG. 4A depicts an endoscopic system in use within a patient duodenum to address a foreign body.

FIG. 4B depicts an endoscopic system in use within a patient duodenum to address a foreign body.

FIG. 4C depicts an endoscopic system in use within a patient duodenum to address a foreign body.

FIG. 4D depicts an example of a patient duodenum in an unaltered conformation.

FIG. 4E depicts an example of a patient duodenum with an conformation altered by an example of an endoscopic overtube.

FIG. 5 is a flowchart showing a method of using an endoscopic system.

FIG. 6 is a flowchart indicating a reprocessing method for the treatment instrument.

### DETAILED DESCRIPTION

This application is related to U.S. Provisional Application Ser. No. 63/267,038, filed on Jan. 21, 2022, which is incorporated by reference herein in its entirety. This document describes, among other things, an endoscope for endoscopic procedures (which can refer generally to include endoscopic, arthroscopic, laparoscopic or similar minimally-invasive procedures). The endoscope, referred to herein generally as a cholangioscope, can include a rotary mechanism providing rotary-assisted articulation of a distal tip. The cholangioscope can be used with an endoscopic overtube for guiding the cholangioscope into anatomic locations within the body, such as across the pylorus of a patient stomach. In an approach to endoscopy at a location inferior to a patient pylorus, an auxiliary scope (also referred to as daughter scope, or cholangioscope) can be attached and advanced through the working channel of a “main scope” (also referred to as mother scope or duodenoscope). Here, the main scope can include optics and can be manipulatable via user controls such as to navigate patient anatomy. The main scope can be inserted at a patient esophagus and operated or manipulated to travel towards a patient duodenum. After a distal end of the main scope is placed at or near a target site within the patient, such as by aid of the main scope optics, the auxiliary scope can be fed through a channel of the main scope such as to treat the target site. Several challenges exist with respect to this approach.

First, the components required for mechanical manipulation of the main, mother scope can constrain the working channel. This can restrict the design of the working channel to a relatively small diameter such as about 4.2 millimeters. This, in turn, limits the size of auxiliary, daughter scopes that can be guided to the target site by the main scope for the procedure. Since it can be desirable to pass additional devices, such as one or more baskets, forceps, or lithotripters, through a working channel of the auxiliary scope, this approach can limit provision or providing of certain additional devices to the target site for the procedure.

Second, the above approach can be difficult and require skillful and time-consuming maneuvering of the main scope beyond the pylorus and towards a patient duodenum. This is due to inherent turns and bends in the anatomical route between the esophagus and the duodenum. Since the above approach involves flexible scopes following the natural disposition of the anatomical route, optics and imaging are heavily relied upon by the physician. Thus, the difficulty in navigating various scopes inferior to the patient pylorus and towards the duodenum can reinforce the need for optics included in two separate scopes and, for certain procedures, such as fluoroscopy or other imaging.

The present inventors have realized techniques for endoscopic treatment for difficult-to-reach locations, such as those inferior to a patient pylorus, using a single endoscope. Devices and methods described herein can include a cholangioscope (sometimes designated the auxiliary, daughter scope, as above) for endoscopic procedures without the need for a parent scope (such as a main scope or duodenoscope) to navigate patient anatomy. Devices and methods described herein can enable navigation of patient anatomy using only one optical system included on the cholangioscope. The cholangioscope can be passed through an endoscopic overtube that can include an elevator for direction towards a target patient site. Herein, “elevator” can include an incline, a ramp, or other shape formed in the overtube to re-direct the path of the cholangioscope when pressed against the elevator. The elevator can be formed of a transparent or similar material permitting visible light or other visualization therethrough by optics of the cholangioscope. The endoscope overtube can include an overtube stabilizer for anchoring the overtube to patient anatomy, such as a patient duodenum. Also, the cholangioscope can include a dual-action mechanism, such as including independent rotation and articulation of a distal tip of the cholangioscope, such as to help enable flexible and precise manipulation of the distal tip through a patient anatomical route.

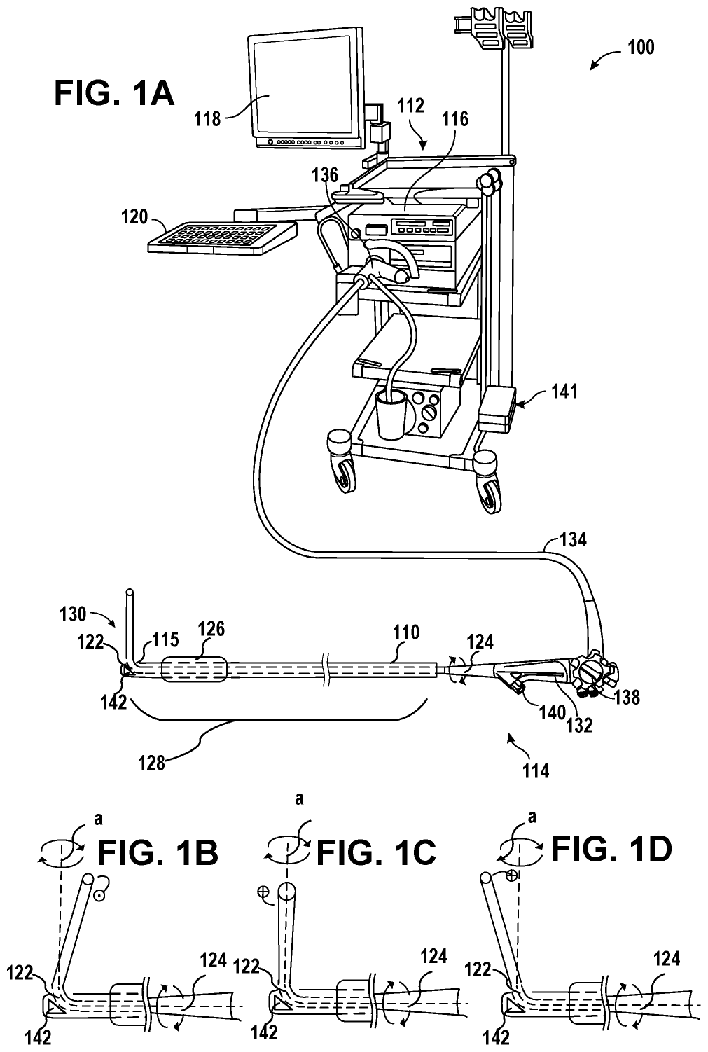

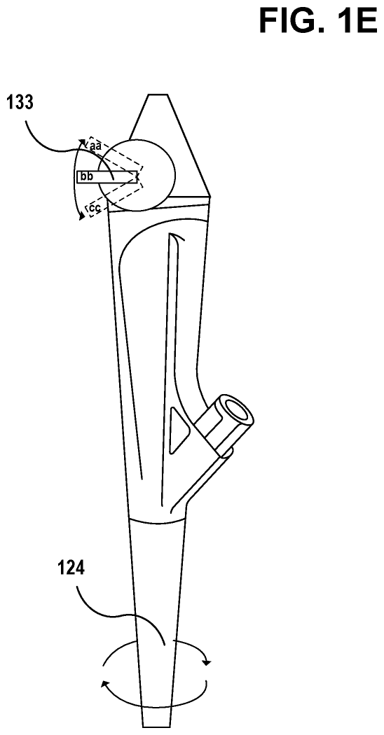

FIG. 1A is a schematic diagram of an endoscopy system 100 including imaging and control system 112, a cholangioscope 114, and an endoscopic overtube 110. The system of FIG. 1A is an illustrative example of an endoscopy system suitable for use with the systems, devices, and methods described herein, such as diagnostic devices or therapy devices. For example, the cholangioscope 114 can be insertable into an anatomical region for imaging and/or to provide passage of one or more sampling devices such as for biopsies, or one or more therapeutic devices for treatment of a disease state associated with the anatomical region. The cholangioscope 114 can interface with and connect to an imaging and control system 112.

The imaging and control system 112 can include or use a controller 116, an output unit 118, and an input unit 120. Imaging and control system 112 can include various ports such as for coupling with the endoscopy system 100. For example, the controller 116 can include a data input and output port for receiving data from and communicating data to the cholangioscope 114. An output unit 118 and an input unit 120 can be used by an operator of the endoscopy system 100 to control one or more functions of the endoscopy system 100 and to view output of the cholangioscope 114. The controller 116 can additionally be used to generate one or more signals or other outputs from treating the anatomical region into which the cholangioscope 114 is inserted. The controller 116 can generate electrical output, acoustic output, a fluid output, or the like such as for treating the anatomical region with, for example, cauterizing, cutting, freezing, or the like.

The cholangioscope 114 can include an insertion portion 128. The insertion portion 128 can include a distal portion 130 and a handle portion 132, which can be coupled to a cable section 134 and coupler section 136.

The insertion portion 128 can extend distally from handle portion 132. The cable section 134 can extend proximally from handle portion 132. The handle portion 132 can be elongate and include a bending portion 122 and a rotating portion 124. The bending portion or joint 122 and the rotating portion 124 can be independently controllable (e.g., by one or more controls 138 on the handle portion 132). This can help to maneuver the distal end through tortuous anatomical passageways (e.g., stomach, duodenum, kidney, ureter, etc.). The insertion portion 128 can also include one or more working channels (e.g., an internal lumen) that can be elongate and can support inserting one or more therapeutic tools of the distal portion 130. The working channel can extend between handle portion 132 and distal portion 130. Additional utilities, such as fluid passages, guide wires, and pull wires can also be provided by the insertion portion 128 (e.g., via suction or irrigation passageways, or the like).

The handle portion 132 can include the one or more controls 138 and one or more ports 140. The one or more controls 138 can include a knob, dial, switch, or other suitable controls for ergonomic actuation by a user. The one or more controls 138 can also be coupled to a pull wire extending through the insertion portion 128. The one or more ports 140 can couple with, e.g., electrical cables, fluid tubes and the like to handle portion 132 for coupling with the insertion portion 128. FIG. 1E depicts an example of a handle portion 132 including a switch or lever 133 for controlling the rotating portion 124. For example, the switch 133 can be a rocker switch including at least three positions, such as a first position “aa”, a second position “bb” and a third position “cc” as depicted in FIG. 1E. For example, the switch 133 can be mechanically biased to a middle, second position bb. In a resting position, such as the middle, second position bb, the switch 133 can not cause rotation of the rotating portion 124. Moving the switch 133 up towards the first position aa can cause the rotating portion 124 to rotate in a first direction. Moving the switch 133 down towards the third position cc can cause the rotating portion 124 to rotate in a second direction opposite the first direction. In an example, releasing the switch 133 from either of the first position aa or the third position cc can allow the switch 133 to return to the second position bb. Several intermediate positions can exist between aa-bb and aa-cc, respectively. For example, moving the switch 133 to an intermediate position between aa-bb or aa-cc can cause respective rotation of the rotating portion 124 at a lesser speed than moving the switch 133 to the first position aa or the third position bb, respectively. As such, the switch 133 can be used both to modulate directionality and rate of rotation of the rotating portion 124

The cholangioscope 114 can be sized and shaped to be received through an access lumen extending through the endoscopic overtube 110. The overtube 110 can include an elevator or ramp 142 in its inner channel at or near a distal tip of the overtube 110. The elevator 142 can be arranged to re-direct the distal portion 130 of the cholangioscope 114 from a longitudinal direction in the access lumen toward the lateral exit of a side port of the overtube 110 upon the cholangioscope 114 being passed through the overtube 110. For example, the elevator 142 can re-direct the distal portion 130 of the cholangioscope 114 in a lateral direction pointing between about 80° and about 100° away from a longitudinal axis of the overtube 110. Also, the bending portion 122 can be actuated in addition to the cholangioscope 114 being re-directed by the ramp 142 to achieve a net-redirection in a direction pointing between about 60° and about 120° away from the longitudinal axis of the overtube. The overtube 110 can be semi-rigid, such as providing rigidity sufficient to manipulate the shape of the patient anatomical route. In an endoscopic system, the overtube 110 can be more rigid than the cholangioscope. For example, the overtube 110 can be placed along the patient anatomical route to help straighten one or more bends included in a natural disposition of the patient anatomy. For example, the rigidity provided by the overtube 110 can provide a splinting of the pylorus to pull the anatomy into a straighter conformation. The overtube 110 can also have flexibility sufficient to pass through the pylorus and to direct the cholangioscope 114 into a patient duodenum.

The overtube 110 can include the port 115 at a side of a distal end of the overtube 110. The aperture can guide the cholangioscope 114 passing through the overtube 110 such as to turn into the biliary anatomy passively, thereby helping to obviate or lessen the need for the physician to utilize the articulation mechanism and rotary mechanism to make the turn. The passive turn can help reduce mechanical stress and wear on the cholangioscope 114.

In an example, the overtube 110 can include or use an overtube stabilizer. The overtube stabilizer can include a stabilizing balloon 126, such as can be located in an area toward the distal tip of the overtube 110. For example, the stabilizing balloon 126 can be inflated to a diameter large enough to create sufficient to contact the inner walls between the stabilizer and the duodenum. In an example, the stabilization balloon 126 can be inflatable to a burst pressure between about 0.5 Atm and about 6 Atm. Also, the stabilization balloon 126, in an inflated state, can have an outer diameter between about 15 mm and about 40 mm. In an example, the stabilization balloon 126 can be fluidly connected to an inflation source via an inflation line, and the inflation line can be included in or used by the overtube 110. For example, the inflation line can be integrated inside the overtube 110 such as within the access lumen of the overtube 110. Also, the overtube stabilizer can include a plurality of expandable wings, arms or folds such as for contacting patient anatomy such as to help stabilize the overtube within the anatomical route.

The overtube stabilizer can be actuated to alter between a stabilizing position, wherein the overtube 110 is anchored to the surrounding patient anatomy, and a released position where the overtube 110 is manipulatable within the patient anatomical route. For example, when the distal portion of the cholangioscope 114 is placed in a desired position in the duodenum, the stabilization balloon 126 may be inflated to contact inner walls of the duodenum such as to act as an anchor for the distal portion of the cholangioscope 114.

The inflated stabilization balloon 126 may help inhibit or prevent the distal portion of the cholangioscope 114 from advancing further than desired into the duodenum or to help inhibit or prevent the cholangioscope from unintentionally withdrawing back into the stomach during the procedure. Once the position of the cholangioscope 114 in the duodenum is held with the overtube stabilizer, the distal tip of the cholangioscope 114 can be advanced through the overtube 110 towards the aperature of the overtube 110. The physician may cannulate the pancreaticobiliary anatomy, such as by advancing the cholangioscope 114 out of the aperature of the overtube 110 while steering the cholangioscope 114 by using the articulation mechanism and the rotary mechanism on the external handle. In an example, the guidewire can be advanced through the overtube 110 such as to help facilitate ductal cannulation.

FIG. 1B, FIG. 1C., and FIG. 1D depict an example of a sequence of events to illustrate the rotation action of the cholangioscope. The cholangioscope 114 can include a dual-action mechanism such as for providing rotation of a distal portion 130 (via the rotating portion 124) independent of articulation of the distal portion 130 (via bending of the joint 122 and the elevator 142). Separate control of a) the articulation angle of the distal portion 130 and b) the radial plane in which the articulation is pointing can help enable a physician to place the distal portion 130 near a target site with precision. The chronologic sequence depicted in FIG. 1B, FIG. 1C, and FIG. 1D shows clockwise rotation of the distal portion 130 for a given articulation. In other words, the sequence shows isolated movement of the rotation portion 124 about a rotary axis a with static articulation. As depicted in the exemplary sequence, the rotary axis a is bent at the articulation angle and the rotation portion 124 is located proximally from the joint 122 and the elevator 142. In another example, the rotating portion 124 can be distal from the joint 122 and the elevator 142, and the rotary axis a can be more straight and not bent at the articulation angle. As the distal portion 130 is rotated on the rotary axis a from FIG. 1B to FIG. 1C, the distal portion 130 travels in a direction out of the page and moves from right to left. As the distal portion 130 is rotated on the rotary axis a from FIG. 1C to FIG. 1D, the distal portion 130 travels in a direction into the page and moves from right to left. As the distal portion 130 is rotated on the rotary axis a beyond FIG. 1D, distal portion 130 travels in a direction into the page and moves from left to right. Thus, for a single given articulation, the distal portion 130 follows a conical path around the rotary axis a. For several articulations, the articulations spanning from small (acute) to large (perpendicular), the distal portion 130 follows several respective conical paths around the rotary axis a. In an example, the possible paths of the distal portion 130 combining rotations and articulations can travel approximately a hemisphere for a given orientation of the overtube 110. In an example, the possible paths of the distal portion 130 combining rotations and articulations of the distal portion 130 as well as rotation of the overtube 110 along the longitudinal axis can travel approximately full sphere around the joint 122, and the radius of the sphere can be approximately the length of the distal portion 130 between a distal tip and the joint 122. Such flexible and controllable motion of the distal portion 130 can help allow the cholangioscope 114 to be maneuvered without needing a second endoscope (such as a parent duodenoscope) or without needing additional visualization or optics to that included in the cholangioscope 114.

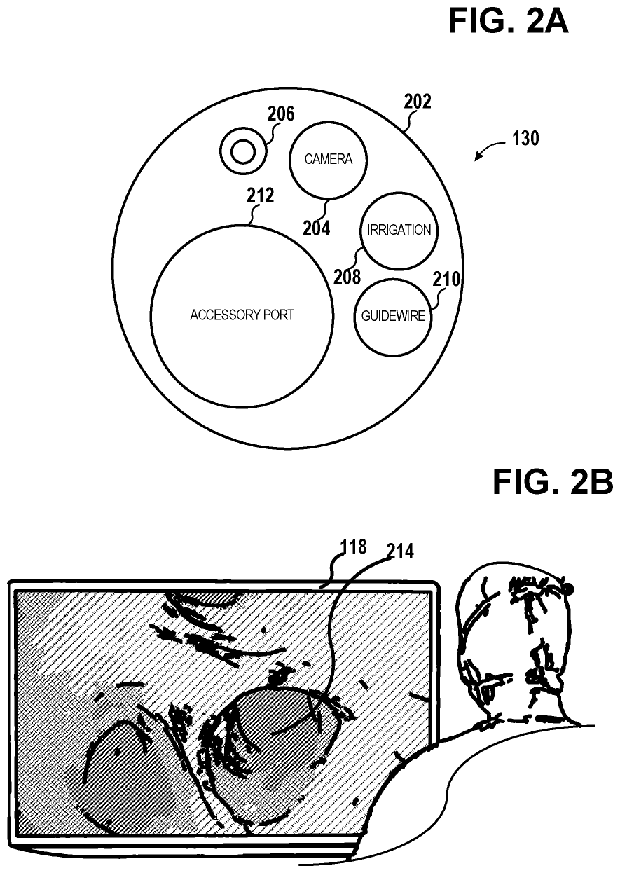

Returning to FIG. 1A, the imaging and control system 112 can be provided on a mobile platform (e.g., cart 141) such as with shelves for housing components such as the controller 116. Alternatively or additionally, one or more components of imaging and control system 112 can be provided directly on cholangioscope 114. The distal portion 130 can include components for treating and diagnosing anatomy of a patient. The distal portion 130 can include an imaging device, an illumination device, and an elevator or ramp 142, such as is described further with reference to FIG. 2A. In an example, the distal portion 130 can include or use one or more electrodes electrically connected to handle portion 132 and also connected to imaging and control system 112 such as to analyze one or more patient anatomy sensors based on comparative biological data stored in imaging and control system 112.

FIG. 2A depicts a portion of an example of a cholangioscope. Referring to FIG. 2A, a distal tip 202 of a cholangioscope can include an optics unit 204, a light source 206, a port for irrigation 208, a guidewire port 210, one or more accessory ports 212 for insertion of accessory devices such as diagnostic or therapeutic devices, or a combination thereof. The optics unit 204 can include a camera that has at least one electronic image recorder that can be arranged to capture a tissue image. The optics unit 104 can include or use refractive optics such as one or more lenses. At least one of the lenses can be mechanically moveable such as to alter a focal length for focusing. The optics unit 104 can include or use at least one sensor such as a direct bandgap compound semiconductor photodetector, a complementary metal oxide semiconductor (CMOS), a charge-coupled device (CCD), a n-channel or other metal oxide semiconductor (NMOS), or other sensor suitable for digital imaging of visible and non-visible wavelengths. Multiple sensors can be concurrently used such as to obtain multiple images concurrently for analysis. Further, the optics unit 104 can include or use an illuminator, wherein this can be generated within the camera module or introduced from outside the camera unit for supplying visible or non-visible light (such as light source 206).

The optics unit 204 can be communicatively coupled with the controller 116 for visualization on the output unit 118 (as depicted in FIG. 1A). In an example, the diameter of the distal tip 202 of the cholangioscope can be between about 4.0 mm and about 8.0 mm. The diameter of the distal tip 202 of the cholangioscope can be between about 4.5 mm and about 6.0 mm. The diameter of the accessory port 212 can be between about 1.5 mm and about 4.5 mm. The diameter of the accessory port 212 can be between about 2.0 mm and about 4.0 mm. In an example, a method for accessing anatomy inferior to a pylorus of a stomach can include visually monitoring an anatomical route with only one camera feed, such as from a single camera located on the cholangioscope and without the need for optics from an additional endoscope or additional imaging.

FIG. 2B illustrates an example of an electrosurgical system in use by a physician. As previously described, the optics unit 204 of the distal tip 202 of the cholangioscope can be communicatively coupled with the controller 116 for visualization on the output unit 118. The optics unit 204 can be capable of capturing a tissue image. For example, the camera can provide a real time, inline, or otherwise concurrent video monitoring feed for the physician and can use frames of the feed as feedback to help the physician maneuver the cholangioscope through the anatomical route and towards a target 214. The video monitoring feed can be viewed on the output unit 118 during a procedure. The video monitoring feed can include or use one or more visual or audible indicators such as to provide feedback to the surgeon during the procedure.

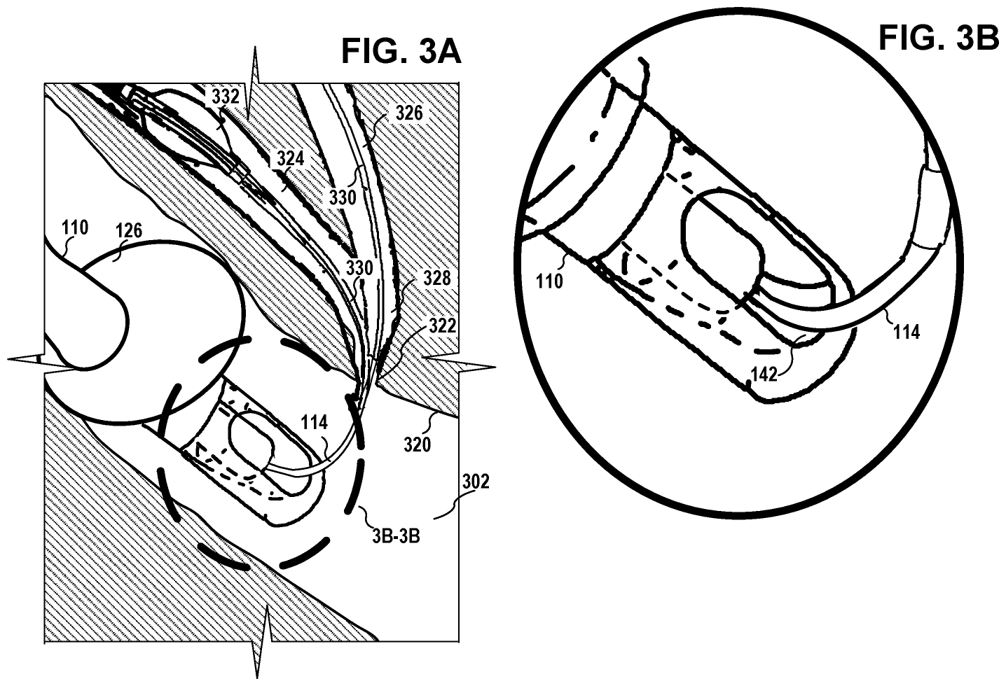

FIG. 3A and FIG. 3B depict a distal portion of an example of an endoscopic overtube 110 positioned in a patient duodenum 302. A patient duodenum 302 can include duct wall 320, sphincter of Oddi 322, common bile duct 324, and main pancreatic duct 326. The duodenum 302 includes an upper part of the small intestine. As previously described, the overtube 110 can include or use an overtube stabilizer such as a stabilization balloon 126, and the stabilization balloon 126 can be inflated such as to contact the duct wall 320 to anchor the overtube 110. Common bile duct 324 carries bile from the gallbladder and liver and empties the bile into the duodenum through sphincter of Oddi 322.

In certain endoscopic procedures, the endoscopic overtube 110 can provide a passage such as to guide the cholangioscope 114 towards or through a patient duodenum 302. As discussed in greater detail below, the cholangioscope 114 can be guided into sphincter of Oddi 322. Therefrom, a physician operating the cholangioscope 114 can navigate the cholangioscope 114 toward the gall bladder or liver to perform various procedures. As such, the surgeon can navigate the cholangioscope past entry 328 of main pancreatic duct 326 and the common bile duct 324. As depicted in FIG. 3A, one or more guidewires 330 can be used such as to cannulate or otherwise access the bile duct 324 or the main pancreatic duct 326. Alternatively or additionally, the cholangioscope 114 can be maneuvered through the sphincter of Oddi 322 without the need to place a guidewire 330 beforehand. In an example, the endoscopic system described herein can enable two guidewires 330 to be placed and used concurrently, such as to allow for cannulization of the main pancreatic duct 326 and the common bile duct 324 at the same time. Also, in an example, a physician can use the cholangioscope 114 to place a stent balloon 332 in one of the main pancreatic duct 326 or the common bile duct 324 and use another therapeutic instrument to concurrently access the other duct.

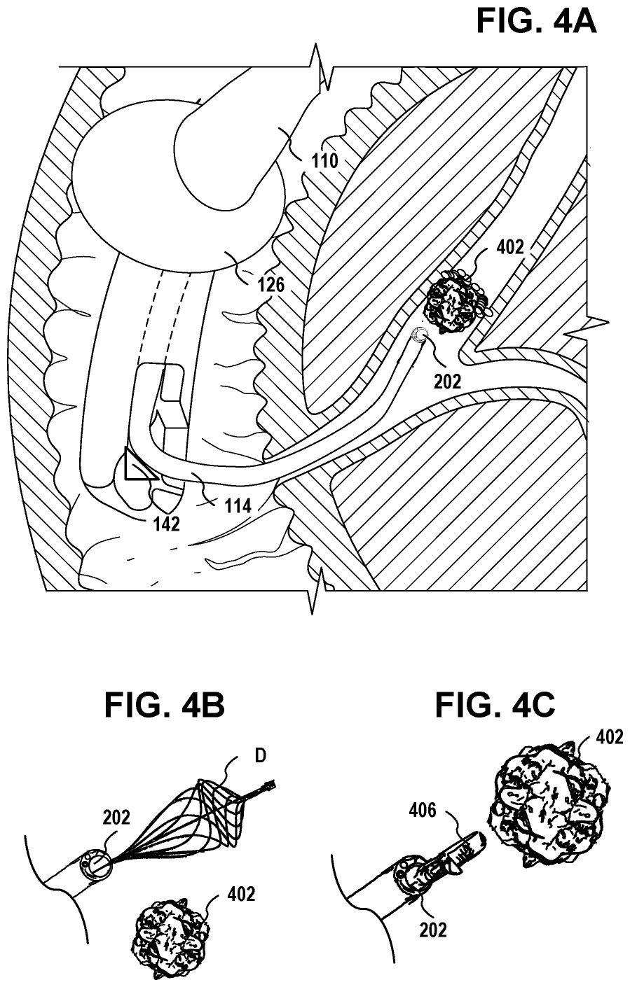

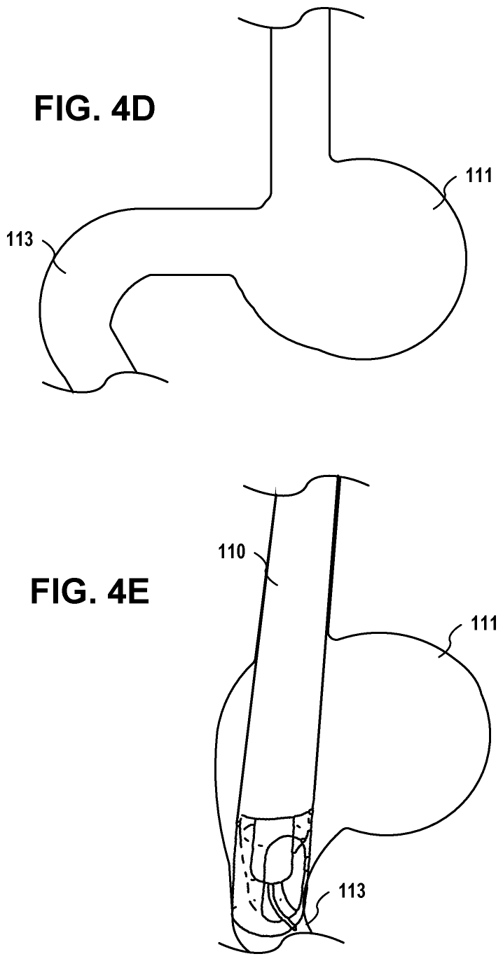

FIG. 4A, FIG. 4B, FIG. 4C, FIG. 4D, and FIG. 4E depict an endoscopic system in use within a patient duodenum to address a foreign body. Cholangioscopes and overtubes described herein can be used such as to provide an access route for an accessory device to the bile duct or the main pancreatic duct. For example, the accessory port 212 of the distal tip 202 of the cholangioscope 114 (as depicted in FIG. 2A) can be sized and shaped such as to receive an ablation catheter, a Through the Scope (TTS) dilation balloon, a TTS stent, a TTS cytology brush, biopsy forceps, lithotripsy probe/filter, or a combination thereof. In operation and use, one or more accessory devices can be used in a procedure such as to address a foreign body 402 located in the bile duct or the main pancreatic duct. First, an endoscopic overtube 110 can be inserted through a patient's mouth, passed down the esophagus, passed through the stomach 111, and passed into the duodenum. In so doing, the semi-rigid endoscopic overtube can help straighten one or more bends included in a natural disposition of the patient anatomy. For example, as depicted in FIG. 4D and FIG. 4E, the endoscopic overtube 110 can be inserted through the stomach 111 and the pylorus 113 such as to splint the pylorus 113 and to pull the anatomy into a straighter conformation. A cholangioscope 114 can then be inserted into an access route of the overtube 110 and guided therethrough. The cholangioscope 114 can be fed into or pressed against a ramp or elevator 142, and the elevator 142 can be used such as to re-direct the distal portion of the cholangioscope from a longitudinal direction in the access lumen toward the lateral exit of a side port of the overtube 110. In an example, the cholangioscope can include optics for visualizing through a material of the elevator 142. For example, the elevator can be formed of a transparent or semi-transparent polymer such as polycarbonate, polyethylene terephthalate (PET), polymethyl methacrylate (PMMA), Amorphous Copolyester (PETG), Polyvinyl Chloride (PVC), and the like. This can help reduce the need for supplemental visualization or imaging. Also, an inner surface of the overtube 110 can include at least one visualization feature to assist manipulation of the cholangiocope 114 through the access lumen of the overtube 110. For example, the overtube 110 can include a line, dots, etching, knurling, or one or more other features able to be detected or identified by cholangioscope optics and in order to help a physician manipulate the overtube without the need for supplemental visualization or imaging. One or more controls can be accessed on a handle of the cholangioscope such as to actuate a rotary mechanism and an articulation mechanism of the cholangioscope, such as independent of one another. The cholangioscope can be manipulated via the rotary mechanism and the articulation mechanism such as to be passed through the sphincter of Oddi, and into a biliary or pancreatic duct. In an example, the bile duct or the main pancreatic duct can be analyzed or visualized via optics of the cholangioscope. A foreign body 402 located within the bile duct or the main pancreatic duct can be located via optics of the cholangioscope. As depicted in FIG. 4B and FIG. 4C, at least one accessory device can be passed through the accessory port of the cholangioscope to address the foreign body 402. In an example, a basket can be passed through the accessory port and used such as to capture or withdraw at least a portion of the foreign body 402. Also, a forceps 406 can be passed through the accessory port 212. The forceps 406 can be manipulated between a first position wherein jaws of the forceps are located further apart and a second position wherein jaws of the forceps are moved closer together. The forceps 406 can be manipulated between the first and second position such as to grab, fragment, cut, cauterize, or energize the foreign body 402. In an example, the forceps can be manipulated to grab at least a portion of the foreign body 402 and withdraw the portion through the accessory port 212.

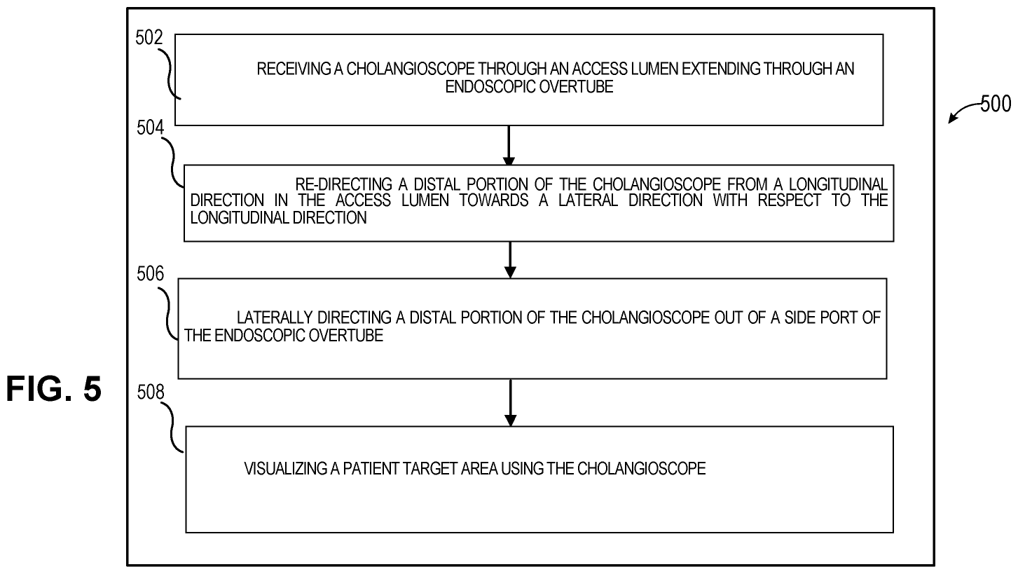

FIG. 5 is a flowchart showing a method of using an endoscopic system. At 502, a cholangioscope can be received such as through an access lumen extending through an endoscopic overtube. For example, the overtube can be sized and shaped such as for insertion into a patient esophagus toward a patient duodenum. At 504, A distal portion of the cholangioscope can be redirected from a longitudinal direction in the access lumen towards a lateral direction with respect to the longitudinal direction, such as by using a ramp located at a distal portion of the overtube. At 506, a distal portion of the cholangioscope can be laterally directed out of a side port of the endoscopic overtube. Also, the distal portion can be articulated. For example, at a particular user-rotation, angular travel of the distal tip can be restricted to an individual longitudinal plane tangent to the distal tip of the cholangioscope. And, at 508, a patient target area can be visualized such as by using the cholangioscope. For example, the patient target area can be visualized the ramp and in the longitudinal direction.

In an example, a guidewire can be placed inside patient anatomy inferior to a pylorus of a stomach of a patient. In an example, a second, different endoscope can be initially inserted to the patient anatomy to place the guidewire, and then the different scope can be removed from the patient anatomy, such as a duodenoscope used for an ERCP procedure. Following removal of the second endoscope, one of the cholangioscope and the overtube can be inserted over the guidewire. In another example at least one visualization feature included on the inside of the overtube can be oriented, such as via cholangioscope optics. In an example, a substantially unbent access route can be provided further comprising providing through the access lumen of the overtube extending between the pylorus and an ampulla of vater of the patient for the cholangioscope via the overtube. Also, the duodenum can be traversed such as with the distal tip of the cholangioscope.

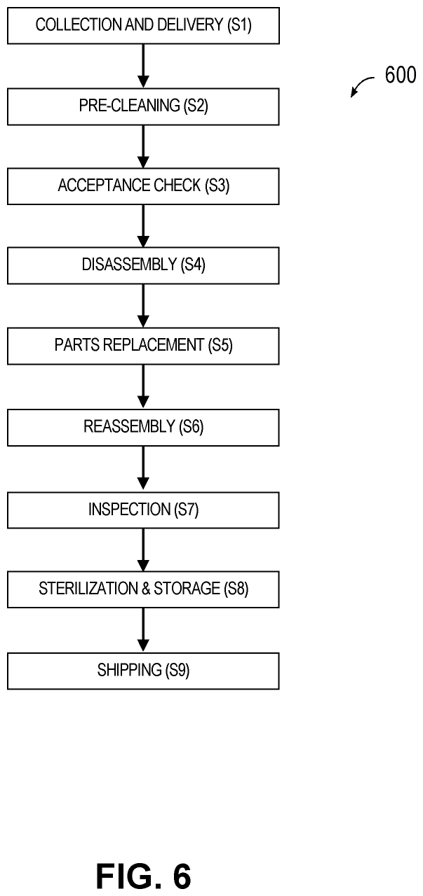

FIG. 6 is a flowchart indicating a reprocessing method for the treatment instrument. A treatment instrument, such at least a part of the cholangioscope 114 or the endoscopic overtube 110 (depicted in FIG. 1) described above may be disposed of after one use, or may be repeatedly used a plurality of times. In the case of a configuration that is repeatedly used a plurality of times, for example, reprocessing method shown in FIG. 6 is required. An operator collects the used treatment instrument after it has been used for treatment and transports it to a factory or the like (Step S1). Then, the operator cleans and sterilizes the collected and transported used treatment instrument (Step S2). Next, the operator performs an acceptance check of the used treatment instrument (Step S3). Subsequently, the operator disassembles the used treatment instrument (Step S4) and replaces some parts of the used treatment instrument with new parts (Step S5). After step S5, the operator assembles a new treatment instrument (Step S6). In some examples, Step S6 can include adding an identifier to indicate the device has been modified from its original condition, such as a adding a label or other marking to designate the device as reprocessed, refurbished or remanufactured. After Step S6, the operator sequentially performs an inspection (Step S7), sterilization and storage (Step S8), and shipping (Step S9) of the new treatment instrument. For example, an endoscopic overtube described herein can include fewer moving parts, electronics, or mechanisms than a conventional duodenoscope. Therefore, there is an advantage that the the endoscopic overtube may need less attention to, or permit omission of parts replacement (Step S5) and reassembly (S6).

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1.-23. (canceled)

24. An endoscopy system for accessing anatomy inferior to a pylorus of a stomach of a patient, the system comprising:
an endoscopic overtube, sized and shaped for insertion into a patient esophagus towards a patient duodenum, the overtube including a proximal portion and a distal portion, and an access lumen extending therebetween, the access lumen providing an inner diameter to receive and pass a cholangioscope therethrough;
wherein:
the distal portion of the overtube includes a side port sized and shaped to permit lateral exit of a distal portion of the cholangioscope; and
the overtube includes a ramp arranged to re-direct the distal portion of the cholangioscope from a longitudinal direction in the access lumen toward the lateral exit of the side port, the ramp formed of a material permitting visualization therethrough by optics of the cholangioscope.

25. The system of claim 24, further comprising a stabilizer disposed about an exterior of a distal portion of the overtube, the stabilizer configured to stabilize the distal portion of the overtube with respect to the duodenum.

26. The system of claim 25, wherein the stabilizer configured to be expanded at the distal portion of the overtube.

27. The system of claim 24, wherein the system includes a guidewire to facilitate the overtube and the cholangioscope to a target anatomy/pancreaticobiliary anatomy.

28. The system of claim 24, further comprising the cholangioscope and the overtube is more rigid than the cholangioscope.

29. The system of claim 28, wherein the cholangioscope includes:
a distal tip that is user-rotatable about a longitudinal axis of the cholangioscope and user-articulatable via separate user controls at a proximal portion of the cholangioscope; and
an elongate tubular member extending between the proximal portion and the distal tip of the cholangioscope.

30. The system of claim 29, wherein the elongate tubular member has an outer diameter greater than 4.5 mm.

31. The system of claim 29, wherein, for a particular user-rotation, adjusting user-articulation of the distal tip of the cholangioscope, when extended out from the side port of the overtube, restricts angular travel of the distal tip to a particular longitudinal plane tangent to a distal end of the elongate tubular member.

32. The system of claim 31, wherein the cholangioscope includes a working channel lumen extending through the elongate tubular member, wherein user-rotation of the distal tip is actuated via a rotary user control mechanism including or connected to a drive to rotate at least a portion of the elongate tubular member around a longitudinal axis of the elongate tubular member.

33. The system of claim 32, wherein the working channel lumen of the endoscope has a diameter greater than 2 mm along a length of the channel.

34. The system of claim 32, wherein the working channel lumen has a large enough diameter to accommodate one or more accessory device including an ablation catheter, a TTS dilation balloon, a TTS stent, a TTS cytology brush, biopsy forceps, or a lithotripsy probe or filter.

35. The system of claim 24, wherein the overtube is configured to provide a substantially unbent access route through the access lumen for the cholangioscope to traverse a patient duodenum within a portion of the overtube extending a distance between the pylorus and an ampulla of vater of the patient.

36. The system of claim 24, wherein:
further comprising an expandable stabilizer includes an inflatable balloon configured to stabilize the overtube with respect to the patient anatomy, wherein the overtube extends through a central lumen defined by the balloon and is attached thereto.

37. The system of claim 36, wherein the balloon is inflatable to a burst pressure above at least 6 Atm.

38. The system of claim 36, wherein the balloon, in an inflated state, has an outer diameter between 15 mm and 40 mm.

39. An endoscopy system for accessing anatomy inferior to a pylorus of a stomach of a patient, the system comprising:
an endoscopic overtube, sized and shaped for insertion into a patient esophagus toward a patient duodenum, the overtube including a proximal portion and a distal portion, and an access lumen extending therebetween, the access lumen providing an inner diameter to receive and pass a cholangioscope therethrough;
wherein:
the distal portion of the overtube includes a side port sized and shaped to permit lateral exit of a distal portion of the cholangioscope;
the overtube includes a ramp arranged to re-direct the distal portion of the cholangioscope from a longitudinal direction in the access lumen toward the lateral exit of the side port; and
an expandable stabilizer is included about an exterior of a distal portion of the overtube, the stabilizer configured to be expanded to stabilize the distal portion of the overtube with respect to the duodenum.

40. The system of claim 39, wherein an inner diameter of the endoscopic overtube is greater than 4 mm.

41. A method for accessing anatomy inferior to a pylorus of a stomach of a patient using an endoscope, the method comprising:
receiving a cholangioscope through an access lumen extending through an endoscopic overtube, the overtube sized and shaped for insertion into a patient esophagus toward a patient duodenum;
using a ramp located at a distal portion of the overtube, re-directing a distal portion of the cholangioscope from a longitudinal direction in the access lumen towards a lateral direction with respect to the longitudinal direction;
laterally directing a distal portion of the cholangioscope out of a side port of the endoscopic overtube;
visualizing, through the ramp and in the longitudinal direction, a patient target area using the cholangioscope.

42. The method of claim 41 further comprising:
placing a guidewire inside patient anatomy inferior to a pylorus of a stomach of a patient;
removing a second endoscope from the patient anatomy; and
inserting one of the cholangioscope and the overtube over the guidewire.

43. The method of claim 41, further comprising orienting, via cholangioscope optics, at least one visualization feature included on an inner surface of the overtube.

44. The method of claim 41, wherein articulating includes, at a particular user-rotation, restricting angular travel of a distal distal tip of the cholangioscope to an individual longitudinal plane tangent to the distal tip of the cholangioscope.

45. The method of claim 41, further comprising providing for the cholangioscope, via the overtube, a substantially unbent access route through the access lumen of the overtube extending between the pylorus and an ampulla of vater of the patient; and
traversing, with a distal tip of the cholangioscope, a duodenum of the patient.

46. The method of claim 41, further comprising extending, through a working channel of the cholangioscope, one or more accessory devices including an ablation catheter, a TTS dilation balloon, a TTS stent, a TTS cytology brush, biopsy forceps, or lithotripsy probe or filter.
